(19) United States
(12) Patent Application Publication
(10) Pub. No.: US 2015/0011864 A1
(43) Pub. Date: Jan. 8, 2015

(54) Prevention of Mild Cognitive Impairment and Eventual Alzheimer's Disease

(71) Applicant: New York University, New York, NY (US)

(72) Inventor: Barry Reisberg, New York, NY (US)

(21) Appl. No.: 14/375,387

(22) PCT Filed: Jan. 29, 2013
(86) PCT No.: PCT/US13/23650

§ 371 Date: Jul. 29, 2014

Related U.S. Application Data
(60) Provisional application No. 61/592,336, filed on Jan. 30, 2012.

(51) Int. Cl. A61K 38/28; A61B 5/0476; A61K 45/06; A61B 5/00; A61K 38/25; A61N 1/36; A61B 5/055

(52) U.S. Cl. CPC A61K 38/28; A61N 1/36092; A61B 5/0476; A61B 5/055; A61B 5/4833; A61B 5/4088; A61K 38/25; A61K 45/06
USPC 600/410; 607/3; 600/544; 600/300; 514/11.2; 514/5.9

(57) ABSTRACT

A method for preventing cognitive decline, comprises the steps of identifying, in an individual, a first stage of cognitive decline corresponding to Subjective Cognitive Impairment and administering a predetermined treatment to the individual to inhibit a progression of the individual to a second predetermined stage of cognitive decline or to inhibit progression of cognitive decline within the first stage.

### PRIORITY CLAIM

The present application claims priority to U.S. Provisional Applin. Ser. No. 61/592,336 entitled “Prevention of Mild Cognitive Impairment and Eventual Alzheimer's Disease” filed on Jan. 30, 2012, the entire disclosure of which is incorporated herein by reference.

### BACKGROUND

There is increasing interest in the development of prevention interventions for Alzheimer's disease (AD). For such studies, there is a need for assessment methodologies for subject characterization and outcome. The Global Deterioration Scale (GDS, Reisberg et al., Am. J Psychiatry, 1982) describes three GDS stages prior to dementia in AD. The terminology “mild cognitive impairment” (MCI) was originally coined for GDS stage 3 (Reisberg et al., Drug Dev. Res., 1988). A 1986 estimate of a 7 year mean duration of the MCI stage in AD (Reisberg, Geriatrics, 1986) is consonant with the observed MCI duration reported in subsequent worldwide studies from clinic populations. The GDS also identifies a pre-MCI stage of subjective cognitive impairment (“SCI”, GDS stage 2, also referred to as subjective complaints of cognitive impairment, subjective memory complaints, subjective cognitive decline, self-reported memory complaints, subjective complaints of memory loss, subjective memory impairment, subjective cognitive complaints, subjective memory loss, subjective memory deterioration, subjective cognitive failures, age associated cognitive impairment, age-associated memory impairment, age associated cognitive decline, older adults with cognitive complaints, memory complaints in patients with normal cognition, preclinical subjective cognitive impairment, preclinical subjective memory impairment, patients with subjective complaints, persons with perceived loss of memory ability, subjective cognitive failures, subjective memory deficits, subjective cognitive dysfunction, etc), in which subjective symptoms occur in the absence of objective clinically manifest overt or subtle cognitive deficits. In 1986, we estimated that the SCI stage of eventual AD lasts a mean of 15 years prior to MCI, This temporal estimate was supported by a subsequent 9-year longitudinal study (Prichep, et al., Neurobiol Aging, 2006). Since SCI appears to occur in approximately 25 to 55% of community residing persons aged 65 years or older, and is (a) a source of concern for these persons and (b) a precursor of AD dementia, there is a need for adequate assessment of SCI. Furthermore, there is a need for a system and method that can prevent the onset of Alzheimer's disease and the associated dementia of AD. That is, medications have been approved for the treatment of Alzheimer's disease associated dementia but these medication are only available for an individual who has already progressed to the stages of dementia (mild, moderate, moderately severe, and severe dementia, respectively).

### SUMMARY OF THE INVENTION

The present invention is directed to a method for preventing cognitive decline comprising the steps identifying, in an individual, a first stage of cognitive decline corresponding to Subjective Cognitive Impairment and administering a predetermined treatment to the individual to inhibit a progression of the individual to a second predetermined stage of cognitive decline or to inhibit progression of cognitive decline within the first stage,

### BRIEF DESCRIPTION OF THE DRAWINGS

FIG. 1 shows a chart depicting a typical time course of normal brain aging, mild cognitive impairment associated with eventual Alzheimer's disease and the dementia of Alzheimer's disease;

FIG. 2 shows a chart depicting a duration of the SCI stage according to the invention over a hypothesized duration of 15 years, determined via a longitudinal study of 44 GDS Stage 2 subjects followed for 8.9±1.8 years;

FIG. 3 shows a survival curve plot depicting decline to mild cognitive impairment or dementia for baseline groups having no cognitive impairment (“NCI”) and SCI;

FIG. 4 depicts an outcome over a mean span of two years of a percentage of 98 GDS scale stage 2 subjects (i.e., subjects with SCI at baseline), the outcomes ranging from GDS stage 1 to GDS stage 4, in the selected subjects;

FIG. 5 depicts, for the study of FIG. 4, an estimated mean duration of GDS stage 2, estimated change in GDS stage 2 over 1 year and the actual observed change in GDS stage 2 subjects over 1 year;

FIG. 6 depicts subject details of subjects who remitted to NCI (GDS stage 1) versus subjects who remained SCI (GDS stage 2) or worsened;

FIG. 7 depicts BCRS scale scores at baseline of subjects who subsequently remitted to NCI versus subjects who remained SCI or worsened for the study of FIG. 4;

FIG. 8 depicts Z-scores corresponding to selected QEEG feature for a subject with a baseline in November, 2010, and a follow-up in January, 2012;

FIG. 9 depicts, at baseline, QEEG topographic images of Z-Scores representing the statistical deviation from age expected normal values for each measure set;

FIG. 10 depicts topographic images of Z-scores representing the statistical deviation from age expected normal values for each measure set at a more than one-year follow up to the baseline of FIG. 9,

FIG. 11 depicts, at baseline, source localization (LORETA) images of the maximum abnormalities in the very narrow band frequency spectrum showing the significance of abnormalities; and

FIG. 12 depicts LORETA images of the maximum abnormalities in the very narrow band frequency spectrum showing the significance of abnormalities at a more than one-year follow up to the baseline of FIG. 11.

### DETAILED DESCRIPTION

The present invention is directed to a system and method for determining a progression of mental impairment in a patient prior to the onset of symptoms and signs of dementia and for treating these patients. Specifically, the system and method according to the invention are directed to assessing a patient to determine if the patient exhibits characteristics corresponding to a stage of cognitive decline corresponding to Subjective Cognitive Impairment (“SCI”) which corresponds to a GDS 2 state. Identification characteristics indicative of the GDS state apart from the Global Deterioration Scale per se, and the GDS staging system methodologies include prognostic markers, as described in greater detail in U.S. application Ser. No. 12/134,768 filed on Jun. 6, 2008 and entitled “Stage Specific Prognostic in Vivo Markers of Brain Aging and Dementia”, the entire disclosure of which is hereby incorporated by reference in its entirety. It is noted, however, that any other diagnostic systems and methods may be employed without deviating from the scope of the invention. The system and method according to the invention further comprises the administration of treatment during the GDS 2 state to prevent further cognitive decline of the patient to a GDS 3 state corresponding to MCI or decline to a dementia status. The system and method according to the invention highlights the utility of pharmacological treatments including antidepressants and other neurogenesis enhancer treatment in achieving remission in SCI and in the prevention of MCI, an at-risk stage for the development of Alzheimer's disease. In one exemplary embodiment of the invention, the treatment employs neurogenesis enhancers configured to increase neurogenesis in the hippocampus, as will be described in greater detail later on. Thus, once the subject has been diagnosed as being in an SCI state, the subject is treated using pharmacological agents specifically targeted to slow/inhibit the progression of the pathology of the SCI stage or cause a remission thereof, as will be described in greater detail later on.

In one embodiment, the system and method according to the invention are directed to the identification of SCI in an individual. Specifically, a two year prospective study was performed to determine SCI outcome. 90 out of 98 subjects involved in the study remained in SCI or progressed while 8 subjects did not progress and rather, remitted to a no subjective or objective cognitive impairment status (“NCI”). It has been found that the Hamilton Depression Scale score at baseline of the remitted individuals was significantly associated with subsequent remission. More specifically, subsequent remitters have significantly lower Hamilton Depression Scale scores at baseline than non-remitters. Thus, in accord with the teachings of the present application, administering neurogenesis enhancers (e.g., antidepressants, etc.) to an individual during SCI can be used to treat, delay progression of SCI and to induce remission in SCI.

Studies performed in accord with the present invention, as outlined in greater detail below, were directed to determining (1) whether specific neurogenesis enhancers can slow or reverse the progression of pathology in the SCI stage, (2) whether antidepressant medications, a class of neurogenesis enhancers, can slow the progression of pathology in the SCI stage.

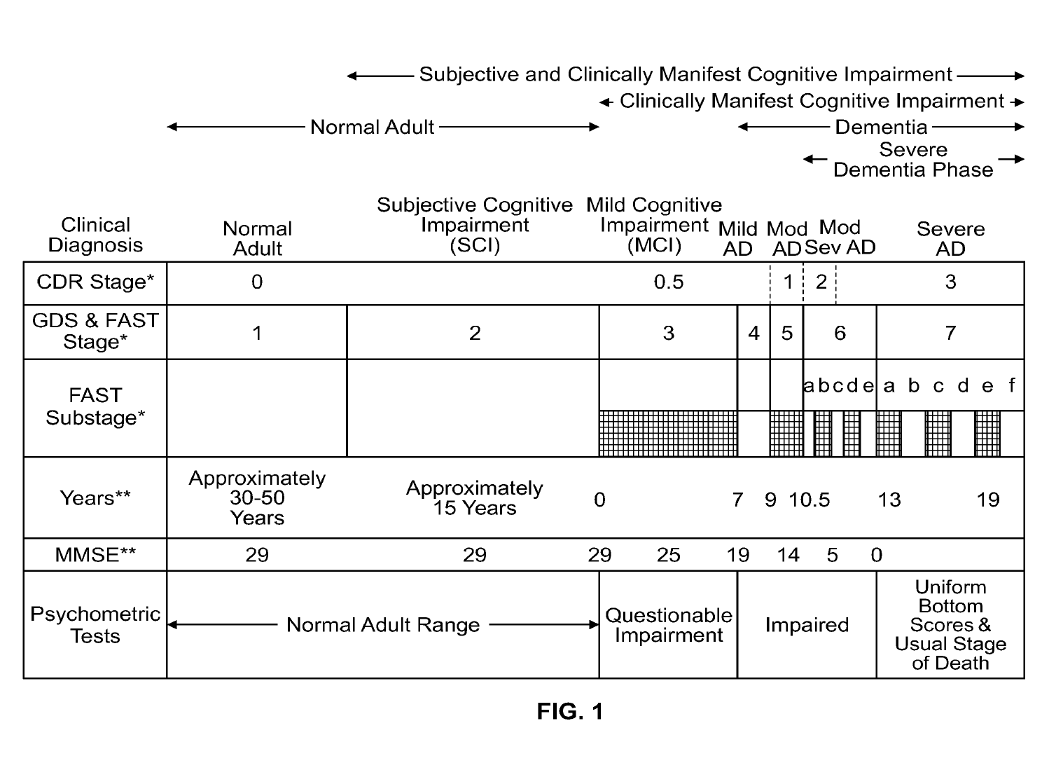

A first two year, prospective study of global and multi-axial assessment of SCI symptomatology is discussed hereinafter. In 1982 we published the Global Deterioration Scale (GDS) which described 7 major stages in the evolution of normal brain aging and progressive Alzheimer's disease (Reisberg, et al., Am J Psychiatry, 1982). Importantly, 3 of the 7 GDS stages occur prior to the advent of overt dementia. In 1986, we published detailed estimations of the duration of the GDS stages and the associated elements of the GDS staging system, the Brief Cognitive Rating Scale (BCRS) and the Functional Assessment Staging procedure (FAST). This 1986 publication (Reisberg, Geriatrics, 1986) estimated a duration of approximately 7 years for the GDS 3 stage, for which we coined the terminology of Mild Cognitive Impairment (MCI) in 1988 (Reisberg, et al., Drug Development Research, 1988). This 1986 publication also estimated that the pre-MCI stage, now termed SCI, lasts approximately 15 years prior to the advent of MCI. The temporal estimates for these stages in the evolution of brain aging and AD are shown in FIG. 1. Specifically, numerical values depicted in FIG. 1 correspond to a time in years. For GDS and FAST Stage 1, the temporal values are determined subsequent to the onset of adult life, For GDS and FAST Stage 2, the temporal values are determined prior to the onset of MCI symptoms. For GDS and FAST Stages 3 and higher, the temporal values are determined subsequent to the onset of MCI symptoms. In all cases, the temporal values refer to the evolution of Alzheimer's disease pathology.

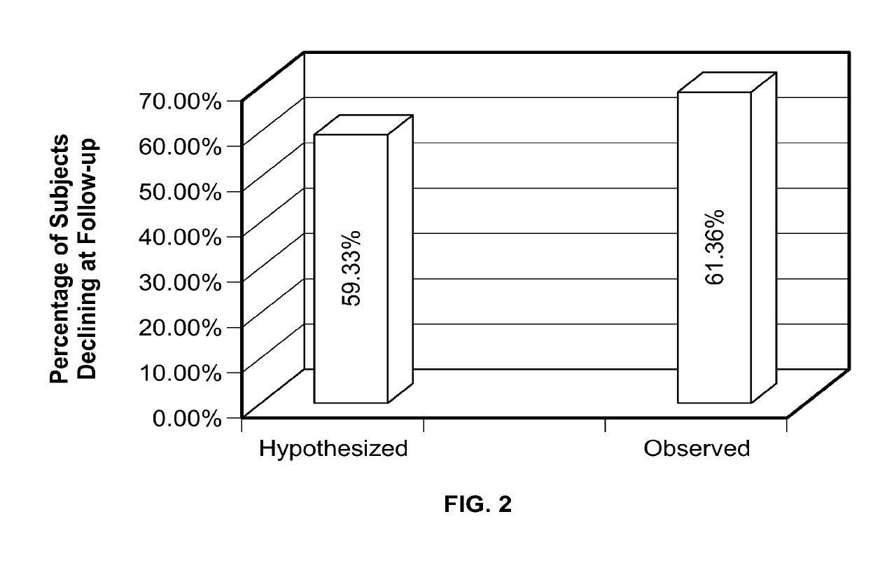

FIG. 2 depicts a chart showing results from a study used to determine a duration of the SCI stage. The results correspond to a longitudinal study of 44 GDS Stage 2 subjects for 8.9±1.8 Years. The hypothesized mean duration of the SCI stage was 15 years. The observed results for subjects declining at follow-up for the SCI stage having a 15 year duration differed from the hypothesized result by only 2% (chart published in Reisberg and Gauthier, International Psychogeriatrics, 2008, from data of Prichep et al., Neurobiology of Aging, 2006). A 2006 study corresponding to a 9 year prospective study of SCI outcome found that 27 of 44 baseline GDS stage 2 (SCI) subjects advanced in GDS stage over the 9 year interval. The resultant close correspondence between the 1986 estimate and the 2006 result is depicted graphically in FIG. 2.

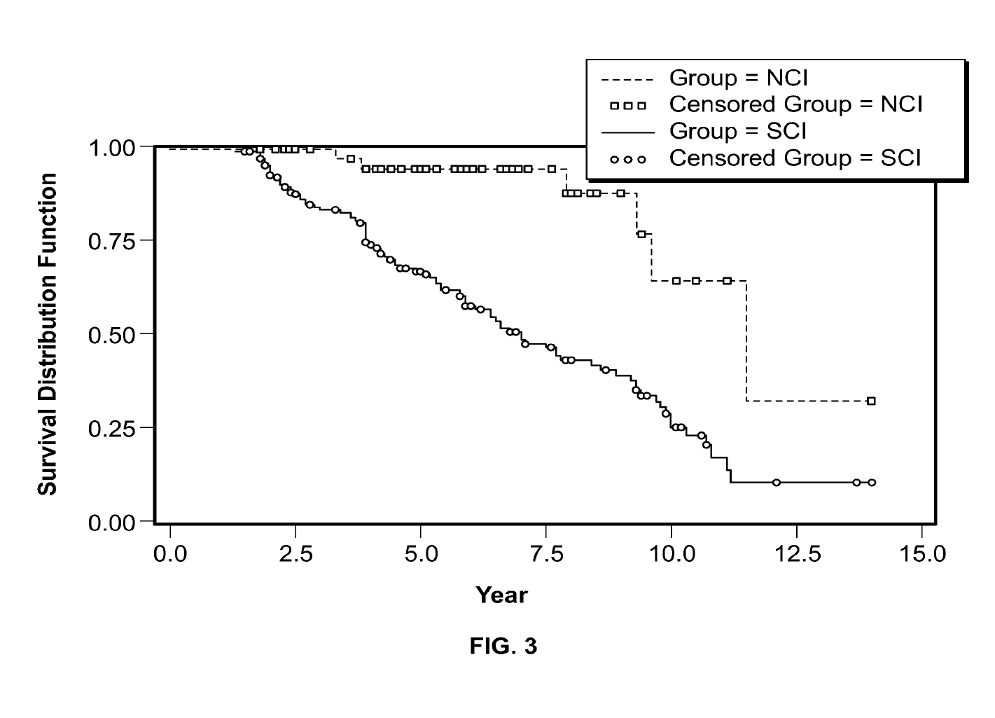

Since pharmacological trials designed to prevent and/or alleviate the progression of cognitive decline in the SCI stage are likely to be of a two year duration, we now describe outcome results at two years from our prior 7 year outcome published study. Specifically, FIG. 3 depicts a plot of survival distribution functions for NCI and SCI baseline groups. The y-axis is the probability of not declining to MCI or dementia, or a survival distribution function. The x-axis is the time (in years) to decline. This survival curve plot extends until observation year 14. There were no SCI subject observations beyond year 14. One NCI subject was followed up beyond year 14. This subject was observed to decline at year 16. There was a significant difference between the NCI and the SCI baseline groups in the survivor function of absence of decline to MCI or dementia in favor of the NCI baseline group (P <0.0001,Wilcoxon test and Log-Rank test) (chart published in Reisberg et al., Alzheimer's & Dementia, 2010) .

SCI is a common condition occurring in a substantial proportion of persons over 65 years of age. The presently disclosed studies indicate physiologic differences between SCI and NCI controls as well as between SCI and MCI subjects. These physiological differences indicate a continuum of pathology from NCI to SCI, to MCI and to AD. Therefore, the present invention seeks to prevent the onset of AD by providing treatment at the early SCI stage.

Studies in accord with the present invention have further identified physiologic markers of the SCI stage which may be utilized to sensitively identify useful treatments. These include quantitative electrophysiological markers (QEEG), electromagnetic markers with LORETA (low resolution electromagnetic topographic analysis), neurometabolic markers using positron emission tomography (PET), and markers using diffusion kurtosis imaging (DKI) of changes in brain structure.

The brain is now understood to exhibit plasticity. Perhaps the most visible aspect of this plasticity is the occurrence of neurogenesis, new neurons, in select brain regions, notably including the dentate gyrus of the hippocampus. The hippocampus is a region prominently involved in AD pathology, showing progressive, linear cell losses with brain aging and AD. Hippocampal neurogenesis declines with aging in mammals and, presumably, man

Depression is associated with a decrease in cognition which can often resemble dementia, a condition formerly termed “pseudodementia” and presently termed the dementia syndrome of depression. Dementia, in this context of depression, can frequently be treated with antidepressants. A history of depression is also associated with an increased risk of Alzheimer's disease. Like aging and Alzheimer's disease, major depression is frequently associated with hippocampal atrophy. This atrophy can persist for several years after remission from the depressive episodes and appears to be related to the duration of the depression episodes. Chronic antidepressant treatment increases neurogenesis in the dentate gyrus of the hippocampus. All classes of antidepressant treatment appear to be associated with an increase in neurogenesis. These include electroconvulsive therapy (ECT) and treatment with the mood stabilizer lithium, as well as treatment with noradrenergic and serotonergic reuptake inhibitors, and even treatment with the atypical antidepressant, tianeptine. Apart from increasing neurogenesis, serotonin and serotonergic antidepressant treatment appears to increase brain plasticity more generally, for example, in the adult visual cortex, as well as plasticity in other regions of the body, apart from the brain, such as the liver.

Apart from the numerous relationships between hippocampal cell loss, neurogenesis, depression, Alzheimer's disease and antidepressant treatment reviewed briefly above, Aβ, an AD related pathology, appears to inhibit neurogenesis. Since neurogenesis appears to be involved in memory and learning processes, or at least those cognitive processes dependent on the hippocampus, the relationship between neurogenesis and AD might, at first glance, appear to be straightforward. However, the relationship between AD and neurogenesis and cognition and neurogenesis, is, in reality, much more complex. Enhanced neurogenesis has been observed in response to various conditions associated with brain injury including seizures, brain ischemia, and traumatic brain injury. Similarly, neurodegenerative diseases have been associated with increased neurogenesis including Huntington's disease and Alzheimer's disease. Especially with respect to AD, this increase in neurogenesis may be viewed in the context of a more general neurodevelopmental response. Since AD is accompanied by continued and continuous cognitive losses as well as continuous hippocampal cellular losses, the effect of the apparent increase in neurogenesis in the dentate gyrus of the hippocampus in AD has been questioned. Recent works indicate that newly generated hippocampal, dentate gyrus neurons do not become mature functioning neurons and furthermore, that this effect is stage dependent. Specifically, in AD, at early stages of neurodegeneration, neurogenesis was significantly enhanced and newly generated neurons migrated into the local neural network. However, at late stages of neurodegeneration, the survival of newly generated neurons was impaired so that the enhanced neurogenesis could not be detected anymore. The studies also concluded that dynamic changes in neurogenesis were correlated with the severity of neuronal loss in the dentate gyrus, indicating that neurogenesis may work as a self-repairing mechanism to compensate for neurodegeneration. Therefore, it is an object of the present invention to enhance neurogenesis at early stages of neurodegeneration as a valuable strategy to delay neurodegenerative progress.

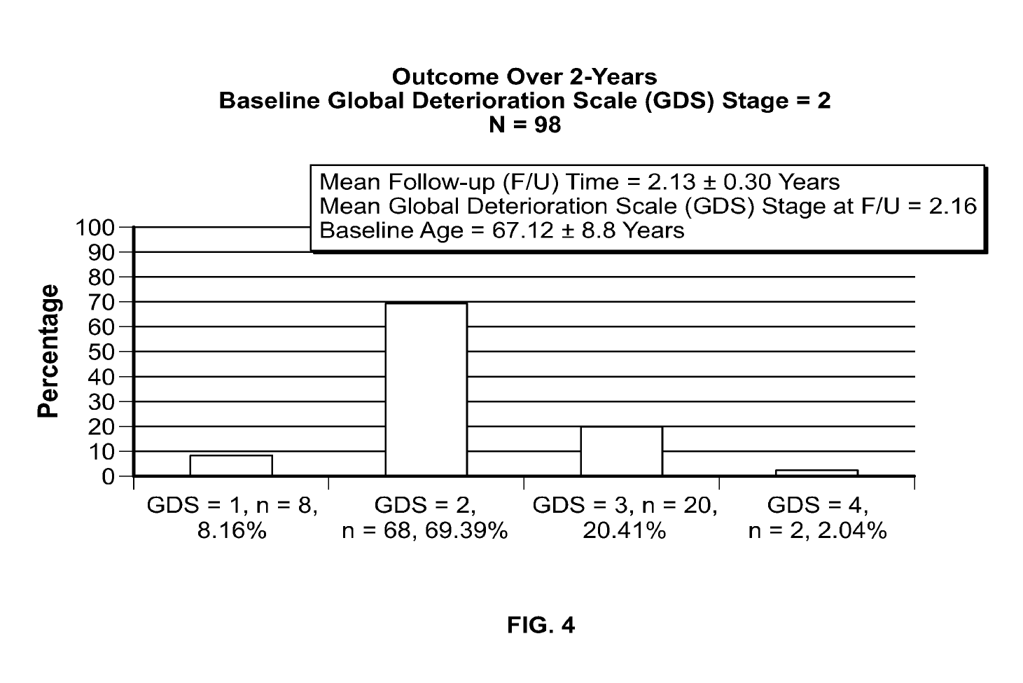

In a further study, subjects from a large, previously reported, 7 year outcome study of SCI and NCI were selected, the selection criteria including: (1) presence of SCI at baseline; (2) otherwise robust health; and (3) a required follow-up 1.5 to 3 years after baseline. Assessments were performed using the GDS and the BCRS, wherein BCRS axes 1 to 5 assess: (1) concentration and calculation; (2) recent memory; (3) remote memory; (4) orientation; and (5) daily functioning. BCRS axis scores are enumerated to be optimally concordant with the corresponding GDS stages. The objective of this study was to examine outcome and stability of SCI after a two year interval. Subjects were selected from a previous longitudinal study population in which 213 subjects fulfilled the selection criteria for robust health and were followed. Of these, 166 subjects had SCI at baseline and were selected for the present study. SCI subjects from the previously published, N=166, 7-year outcome study were selected if they had follow-ups 1.5 to 3.0 years post baseline. This resulted in an N=99 follow-up population. A subject with a baseline Hamilton score>21, n=1 was excluded because of the presence of clinically significant depression. The final “2 Year” cohort studied comprised N=98 subjects, as will be described in greater detail below with respect to FIGS. 4-7.

Results from this study indicate similarities between depression affective disorder and

AD. However these similarities, although important for the diagnosis and differential diagnosis of SCI, MCI and AD, do not imply any clear treatment import. That is, AD and its antecedents are marked by plaques (including the beta amyloid protein as well as other constituents) and tangles (comprised of hyperphosphorylated tau protein as well as other elements) in the brain whereas, depression is not. Furthermore, it is concluded that increased Hamilton scores occur with, among other things, remission from MCI. A large body of prior work from many studies has indicated that persons with majordepressive disorder with Hamilton scores greater than or equal to 21 can be associated with a temporary cognitive disturbance which may remit, or may also be associated with eventual AD. Our N=98 study of remission from SCI in persons free of major or minor depression and free of dysphoria yielded Hamilton depression scale scores for ₜhe total SCI subject population which were very low and which did not meet the criteria of clinical depression. However, our N=98 “2 year” outcome study further indicated that low Hamilton scores at baseline were associated significantly with subsequent remission to NCI status at a two year follow up. Since, by definition, Hamilton depression scale items can be treated with anti-depressants, we now, novelly and unexpectedly, conclude that we can produce remission in SCI by treating with neurogenesis enhancers (e.g., antidepressants, etc.). Our present findings are very different from our prior observations that high baseline Hamilton Depression Scale scores are associated with worsening to Mild Cognitive Impairment and subsequent improvement (Glodzik-Sobanska, Reisberg, De Santi, et al., Dementia and Gereatric Cognitive Disorders, 2007). These would appear to be entirely separate and, indeed, paradoxical observations.

From the prior 7 year outcome study, of 166 SCI subjects, 99 had follow-ups within the selection time-window. Of these, one subject was found to have a Hamilton score of 22 at baseline indicative of significant depression, and was excluded. The 98 studied subjects had a mean age of 67.12±8.8 years, an education level of 15.55±2.6 years, and 64% were women. At baseline, the mean score on the 5 BCRS axes was 1.79±0.33. Hence, as per design, the BCRS axis scores were closely concordant with the GDS stage in this selected study of exclusively GDS stage 2 subjects. Similarly, the mean BCRS score at the two year follow-up for subjects free of deficits (i.e., GDS=1), was 1.10±0.20. This was lower than that of subjects manifesting subjective and/or overt deficits at follow-up, i.e., 1.92±0.50<0.001).

Prior studies had indicated that older persons with subjective cognitive deficits are more likely than those without these deficits to progress in subsequent years to MCI or dementia.

The present study indicates that clinical measures previously applied for pivotal worldwide studies of dementia medications (specifically, the GDS and, for certain analyses, the BCRS, used in pivotal trials for rivastigmine and memantine), can demonstrate statistically significant differences in severity after a two year period in an SCI cohort. Therefore, prevention trials over a two year period, in pre-MCI, SCI subjects, with conventional clinical assessments appear to be feasible at the current time utilizing promising pharmacological interventions.

The N=98 subjects selected for the present study exhibited the following characteristics:

| | |
|---|---|
| Age | 67.12 ± 8.8 years (range = 40-87 years) |
| Gender | 63 Female, 35 Male |
| Education | 15.55 ± 2.6 years |
| GDS Stage | 2 |
| MMSE | 28.92 ± 1.23 |
| Follow-up time | 2.13 ± 0.30 years (range = 1.57-2.93 years) |

FIG. 4 depicts an outcome over a span of two years of a percentage of GDS scale in the selected subjects, wherein the mean follow-up (F/U) time=2.13±0.30 years, the mean GDS stage at F/U=2.16 and the baseline age=67.12±8.8 years. The results of the studies performed herein indicate that, over a two year mean follow-up interval, subjects with SCI showed changes in the GDS in accord with prior estimates and observations in 7 to 9 year prospective observational studies.

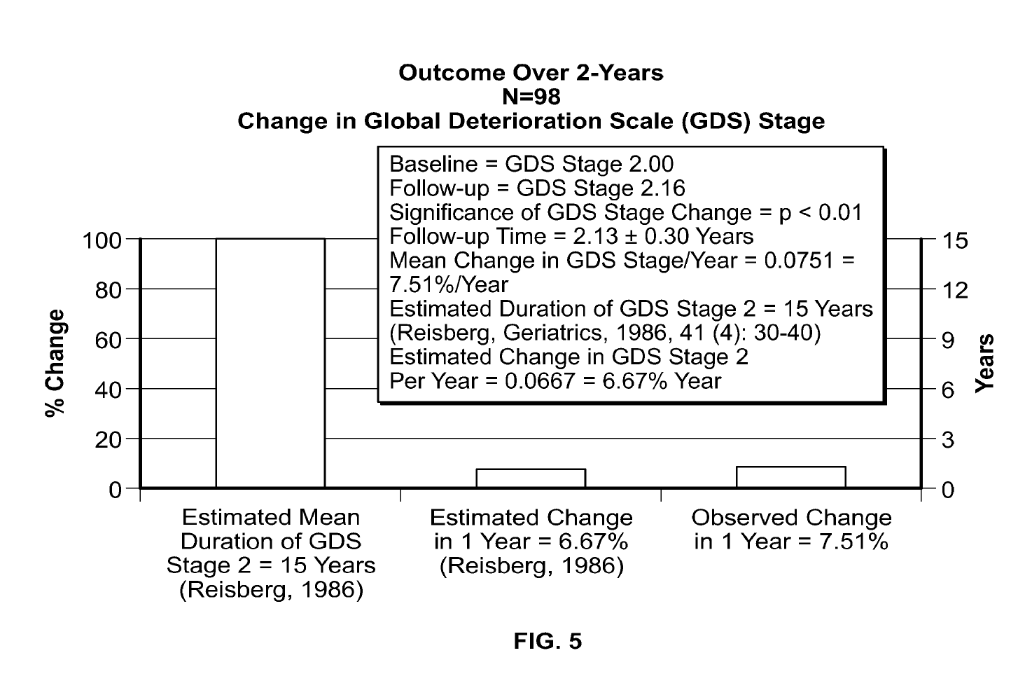

FIG. 5 depicts an outcome over two years depicting a percent change in the GDS, wherein the results indicate that a difference between observed change/year and estimated change/year: 7.51% (observed)−6.67% (estimated)=0.84% , therefore<1% difference. Hence, the progression observed for GDS stage 2 subjects over much longer intervals (e.g., 9 years) can also be observed over an interval of only two years, as evidenced from this data. The change in GDS scores over this two year interval was significant (p<0.01) using the Wilcoxon test. 9 psychometric assessments were undertaken in the 98 SCI subjects over the two year span. There were no consistent changes in the psychometric tests over the 2 year interval. That is, a psychometric deterioration score, which is a combination of the 9 psychometric assessment test scores, was not significantly different at baseline from scores measured at the two year follow up.

The present study also examined a mood, via the Hamilton Depression Scale, of the subjects at the baseline and compared this to corresponding data collected at the two-year follow up. The resultant data indicates that for the entire sample of 98 subjects, mood assessment (i.e., Hamilton Depression Scale scores) significantly improved at the two year follow-up. Specifically, the baseline Hamilton Depression Scores were a mean of 4.52±4.05 (standard deviation) from a total score range of 0 to 16, as those skilled in the art will understand. At the two-year follow up, the Hamilton Depression scores were 3.53±3.51, indicating an improvement (p=0.021).

The present study further examined an outcome over two years of BCRS in the 98 selected subjects. Remote memory assessments corresponding to BCRS axis 3, as those skilled in the art will understand, increased significantly over the two year span for subjects indicating worsening. Other BCRS axes were also examined in the present study, including BCRS axis 1 corresponding to concentration and calculation, BCRS axis 2 corresponding to recent memory, BCRS axis 4 corresponding to orientation and BCRS axis 5 corresponding to functioning and self-care. These axes, however, did not change significantly from baseline measurements over the two year span. Overall, the total BCRS axis Ito 5 score showed a significant decline (p<0.05 using the Wilcoxon test analysis) over the two year interval.

The present study further examined an outcome over two years of MMSE in the selected subjects, which indicated that there were no significant changes in MMSE over the two year interval. Specifically, MMSE measurements at baseline changed from 28.92±1.23 (range of 25-30) at baseline to 28.80±1.70 (range of 19-30) at the two year follow-up.

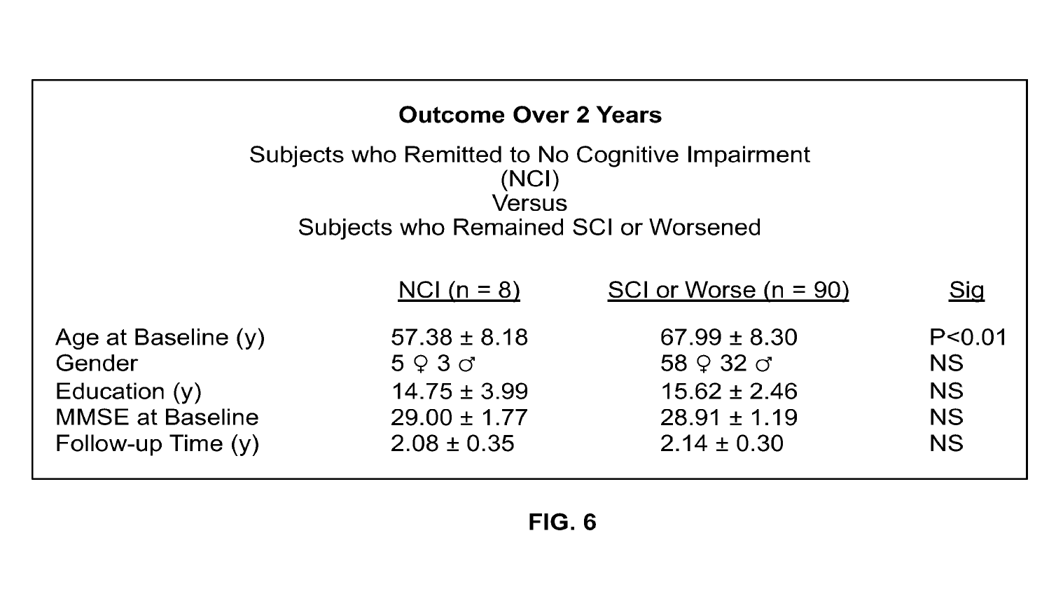

FIG. 6 depicts subject demographic and other details of subjects who remitted to NCI versus subjects who remained SCI or worsened. Subjects who subsequently remitted from SCI to NCI were significantly younger at baseline (p<0.01, Wileoxon analysis). There were no significant differences between the remitters to SCI and the non-remitters in gender or education, or MMSE scores at baseline. There were also no significant differences between the remitters and the non-remitters in the follow up times. No consistent effects were observed on the previously disclosed 9 psychometric assessments or the combinatorial psychometric deterioration score in terms of baseline score differences between subjects who subsequently converted to NCI at the two year follow up and subjects who remained at the SCI stage or deteriorated at the two year follow up.

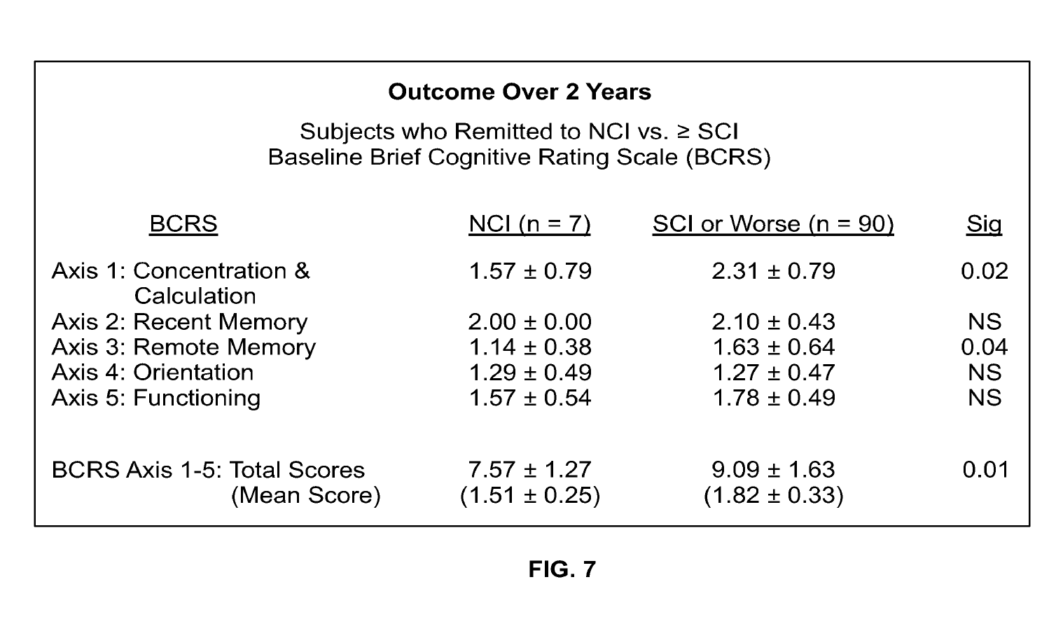

FIG. 7 depicts BCRS scale scores at baseline of subjects who remitted to NCI versus subjects who remained SCI or worsened. One of the 98 subjects studied did not have the BCRS data, Hence this sample consisted of the remaining 97 subjects. Using the Wilcoxon analysis procedure, there were significantly lower (better) scores at baseline in the subjects who subsequently remitted to NCI in comparison with the non-remitting subjects (i.e., subjects who remained SCI or who worsened to an MCI or dementia status) in BCRS axis 1 to 5 total scores (p=0.01). There were also significantly lower baseline scores in the subject group subsequently remitting to NCI on the baseline BCRS Axis 1 and BCRS Axis 3 scores.

For the same study, scores at baseline on the Hamilton Depression Scale were measured for subjects who remitted from SCI to NCI over the two year follow-up study period and for subjects who remained SCI or worsened. Specifically, for the seven subjects remitting to NCI the Hamilton Depression Scale score at baseline was 1.86±2.85. For the ninety subject s who remained in SCI or worsened, the Hamilton Depression Scale score at baseline was 4.72±4.07. It should be noted that the Hamilton scores in both groups are low and are comparable with the range of scores seen in a normal, non-depressed, elderly population. Hamilton scores for major depression are typically 21 or greater. The subjects who remitted to an NCI state at the two year follow-up, had significantly lower Hamilton Depression Scale scores at baseline than the subjects who remained SCI or progressed. We conclude from these results that producing lower depression scores can result in positive outcomes in terms of the process of progression to eventual Alzheimer's disease. The exemplary system and method according to the invention seeks to obtain these low Hamilton depression scores using neurogenesis enhancers such as antidepressants. MMSE scores were also measured for subjects who remitted from SCI to NCI over the two year follow-up study period and for subjects who remained SCI or worsened. Specifically, for eight subjects who remitted to NCI, the MMSE score at baseline was ±1.77. For the ninety subjects who remained in SCI or worsened, the MMSE score at baseline was 28.91±1.19, indicating a non-significant difference between the remitting and the non remitting subject groups on the MMSE at baseline.

Exemplary neurogenesis enhancers according to the invention may include physical activities (e.g., exercise) and/or pharmacological treatments selected from the following list. It is noted that this list is exemplary only and that any other pharmacological treatment may be used without deviating from the scope of the invention.

In one embodiment, the neurogenesis enhancers may be cholinergics, substances that affect the neurotransmitter acetylcholine or the components of the nervous system that use acetylcholine. Acetylcholine is a facilitator of memory formation. Forebrain acetylcholine regulates adult hippocampal neurogenesis. Cholinergic nootropics include acetylcholine precursors and cofactors, and acetylcholinesterase inhibitors including precursors (Choline, precursor of acetylcholine and phosphatidylcholine; DMAE (dimethyl-amino-ethanol), precursor of acetylcholine; Mecclofenox ate, a probable precursor of acetylcholine, approved for dementia and AD), cofactors (Acetylcarnitine, an amino acid that functions in acetylcholine production by donating the acetyl portion to the acetylcholine molecule, Vitamin B5, a cofactor in the conversion of choline into acetylcholine; Huperzine A, which is also shown to act as an NMDA antagonist and appears to increase nerve growth factor levels in rats); Acetylcholinesterase inhibitors, which improve nerve cell regeneration (Donepezil; Rivastigmine; Galantamine), Nicotinic acetylcholine receptor agonists (Nicotine; Epipatidine; Varenicline) and Acetylcholine release stimulators such as ZSET1446/ST101 (spiro[imadazo[1,2-a]pyridine-3,2-indan]-2(3H)-one), which has been shown to both promote hippocampal neurogenesis and also to ameliorate depressive behavior.

In another embodiment, the neurogenesis enhancer may include Dopaminergics, Dopamine Receptor Agonists which include both in vivo and in vitro dopaminergic agonists directed to augmenting subventricular zone cell numbers via a recruitment of D3 receptors. As those skilled in the art will understand, this effect reflects enhanced mitogenesis. Therefore, dopaminergic receptor stimulation is a therapeutic target for neurogenesis, Also D2/D3/D4 receptors influence proliferation of new stem cells and progenitor cells (L Dopa; Pergolide; Pramipexole; Ropinirole; Bromocritine; Carbagoline).

In another embodiment, the neurogenesis enhancers may be glutamate activators which include AMPA transmitters and AMPA receptor agonists, classified as Ampakines, racetams such as aniracetam (also CX516, IDRA-21, LY-503,430), In contrast, AMPA receptor antagonists such as NBQX have negative effects on the brain's ability for neurogenesis and repair. In another embodiment, the neurogenesis enhancers may be any antidepressants. In yet another embodiment, the neurogenesis enhancers may be serotonergic antidepressants which affect the neurotransmitter serotonin or the components of the nervous system that use serotonin. Serotonergic neurogenesis enhancers include serotonin precursors and cofactors, and serotonin reuptake inhibitors or selective serotonin reuptake inhibitors (“SSRIs”) which are a class of antidepressants that increase active serotonin levels by inhibiting reuptake and which have also been shown to promote neurogenesis in the hippocampus (e.g., Citalopram; Escitalopram; Fluoxetine (Prozac, Prozac Weekly, Sarafem); Paroxetine (Paxil, Paxil CR, Pexeva); Sertraline; Scelctium tortuosum—active constituent mesembrine shown to act as an SSRI and PDE4 inhibitor; Hypericum perforatum, which inhibits reuptake of serotonin (as well as norepinephrine, dopamine, GABA and glutamate) via activation of TRPC6), reuptake enhancers or selective serotonin receptor enhancers (“SSREs”) (e.g., Tianeptine, a paradoxical antidepressant which improves mood and reduces anxiety by promoting stress-associated impaired neuroplasticity and enhancing the extracellular concentration of dopamine in the nucleus accumbens and modulating the D2 and D3 dopamine receptors). Still further, the neurogenesis enhancers may include serotonin-norepinephrine receptor inhibitors (“SNRIs”) (e.g., Venlaflaxine, Duloxetine, Levomilnacipran), Tricyclic antidepressants (e.g., Imipramine; Amitryptiline; Nortriptyline; Clomipramine; Doxepin; Desipramine), Tetracyclic antidepressants (e.g., Amoxapine, Maprotiline, Mirtazapine, Mianserin), MAO-B inhibitors (e.g., Tranylcypramine, Phenelzine), Antidepressants in clinical trials (e.g., B2061032, LLY2216684, LUAA21004, BMS820836, SPD489, OPC-34712, B2061014, ALKS5461, ABT-436, EB-1010 (Amitifadine), AZD6765, MK6096 (MK-6096-022AM3), RO4995819, LY2940094, RO4917523, TC-5214 (S-mecamylamine), Pregabalin, Omega-3, JNJ-40411813, FK949E, DVS SR (Desvenlafaxine succinate sustained release), Ketamine, Paliperidone, Vilazodone, Cariprazine, Armodanafil, Mifepristone, Ramelteon, STMS: (Synchronized Transcranial Magnetic Stimulation), GLYX-13, Riluzole, Scopolamine, the triple reuptake inhibitor DOV216, 303, CTN-986 (a compound extracted from cottonseeds which increases hippocampal cell proliferation), the triple reuptake inhibitor (1S, 2S)-3-(methylamino)-2-(naphthalen 2-yl)-1-phenylpropon-1-01 (PRC200-SS), the novel serotonin type 2C receptor inverse agonist/a 2—Adrenoceptor Anagonist S32212, the triple reuptake inhibitor JZAD-IV-22, the metabotropic glutamate 7 receptor agonist N,N-Bis (Diphenylmethyl)-1,2-Ethanediamine (AMN082), Atypical Antidepressants (e.g., Bupropion, Trazodone, Vilazodone), MAO-A inhibitors (e.g., Resveratrol, Curcumin, Piperine, Rhodiola rosea), NMDA receptor antogonists (Amantadine, Memantine, Nitrous Oxide, Phencyclidine, Ethanol, Dextromethorphan, Dextrophan, Ketamine), GABA receptor agonists, wherein GABA is a signal that regulates the speed of neuronal migration during adult subventricular zone neurogenesis (e.g., Valproate, Topiramate, Baclofen, Ethanol, Barbiturates, Benzodiazepines, Zolpidem, Isoflurane, Pentobarbitone, Gabapentin, Lamotrigine), Sildenafil, a phosphodiesterase type 5 (PDE5) inhibitor, Phosphodiesterase-4 inhibitors (e.g., Apremilast, Mesembrine, Rolipram, Ibudilast, Piclamilast, Luteolin, Roflumilast, Cilomilast, Diazepam) and Cyclic AMP (cAMP) and the Cyclic AMP Response Element Binding Protein (CREB) pathways, wherein activation of the cAMP signal transduction cascade increases neuronal differentiation and neurite outgrowth. Antidepressant treatment upregulates the cAMP signal cascade in the hippocampus, possibly mediating the antidepressant neurogenesis effect. Phosphodiesterase-4 inhibitors, such as rolipram also affect the cAMP pathway. These and related studies have demonstrated that activation of the cAMP pathway increases hippocampal granule cell proliferation and the inhibition of CREB decreases this process. In yet another embodiment, the neurogenesis enhancer may be Lithium or moderate ethanol consumption, which increases hippocampal cell proliferation and neurogenesis in adult mice.

In yet another embodiment, the neurogenesis enhancers may be directed to nerve growth stimulation and brain cell protection. Specifically, nerves are necessary to the foundation of brain communication and their degeneracy, underperformance, or lacking can have disastrous results on brain functions. Antioxidants may prevent oxidative stress and cell death, therefore exerting a neuroprotective effect in combination with one or more neurogenesis enhancer compounds. Neurogenesis enhancers according to the invention may therefore also include Idebenone, an antioxidant; Melatonin, an antioxidant; Glutathione, an antioxidant; Acetylcarnitine (Acetyl-L-Carnitine Arginate or Hydrochloride) neuroprotective; Inositol, which is implicated in memory function, with a deficit linked to some psychiatric illnesses and has been shown to be particularly efficacious in OCD patients; Phosphatidylserine, which is a possible membrane stabilizer; Lion's Mane Mushroom, which stimulates myelination and nerve growth factor and improves cognitive ability; SAM-e (S-Adenosyl methionine), which is crucial for cellular regeneration by fueling DNA methylation; Acetylcysteine (L-cysteine), which is a precursor to the antioxidant glutathione; Apoaequorin, a Calcium-binding protein (CaBP), (which is neuroprotective); Uncaria tomentosa (Cat's Claw), which inhibits formation of brain beta amyloid deposits, which have been connected to AD and neurotoxicity.

The neurogenesis enhancers may also include direct hormones such as Pregnenolone sulfate or Thyroxine, which have been shown to be effective in enhancing neurogenesis. The neurogenesis enhancer may further include insulin, other insulin receptor stimulators, growth hormones, IGF-1 (insulin like growth factor 1), IGF-2 (insulin like growth factor 2), growth hormone, growth hormone releasing hormone (also known as growth hormone releasing factor and tesamorelin), insulin like growth factor 1 (IGF-1) receptor stimulators, and insulin like growth factor 2 (IGF-2) receptor stimulators. As those skilled in the art will understand, brain insulin receptors are densely localized in the hippocampus, the entorhinal cortex, and the frontal cortex. These insulin receptors are found primarily in the synapses, wherein insulin signaling contributes to synaptogenesis and synaptic remodeling. Insulin also modulates the levels of Aβ, the pathologic protein of Alzheimer's disease and protects against the detrimental effects of Aβ oligomers on synapses. Neurogenesis enhancers according to this embodiment include rapid acting insulin (e.g., Lispro (Humalog), Aspart (Novolog), Glulisine (Apidra)), short acting insulin (e.g., regular (R), humulin or novolin, velosulin (for use in the insulin pump)), intermediate acting insulin (e.g., NPH (N), Lente (L)), long-acting insulin (e.g., Ultralente (U), Lantus, Levimir or detemir), premixed insulin (e.g., Humulin 70/30, Novolin 70/30, Novolog 70/30, Humulin 50/50, Humolog mixed 75/25) and aerosolized insulin or intranasal insulin. Both exogenous and endogenous GH (growth hormone) and/or IGF-1 may be used as agents to enhance cell genesis and neurogenesis in the adult brain. GHRH (growth hormone releasing hormone) according to this embodiment may include Sermorelin (sometimes referred to as GRF1-29 (Geref)), Tesamorelin (Egrifta) and Ghrelin (Growth Hormone-Releasing Peptide), an agonist at the human growth hormone secretagogue receptor 1a. Growth hormone therapy has been shown to induce cell genesis in the adult brain and may include Somatropin (Norditropin, Nordiflex, Nutropin, Nutropin AQ, Omnitrope, Saizen, Humatrope, Tev-Tropin, Serostim, Nutropin Depot, Accretropin, Genotropin, Nordetropin Flex Pro, Zorbtive) or Sermorelin (Geref). IGF-1 increases progenitor cell proliferation and numbers of new neurons, oligodendrocytes, and blood vessels in the dentate gyrus of the hippocampus and may include IGF-1 Long R3 (Revitropin), IGF-1 (Liposomal spray), IGF-1, IGF-1 DES and IGF-1 LR3. IGF-2 is a mitogenic polypeptide which together with insulin and IGF-1 belongs to the IGF/IGF binding protein system. IGF-2 is the most abundantly expressed IGF in the adult brain. The IGF/IGF binding protein system is important in normal growth and development and tissue repair throughout the life span. In the hippocampus, IGF-2 promotes IGF-2 receptor dependent, persistent long Mini potentiation after synaptic stimulation. Other growth factors such as basic fibroblast growth factor, have also been found to enhance neurogenesis. Basic fibroblast growth factor increased mitotic nuclei in the subventricular zone and the olfactory tract of both neonatal and adult rats.

The sex hormones have also been demonstrated to be neurogenesis enhancers. For example, estradiol has been shown to enhance neurogenesis in the dentate gyri of the hippocampus. Also, 17β-estradiol (E2), the principal mammalian estrogen, and estrone, a common component of hormone replacement therapies, have been shown to impact cell proliferation in the dentate gyrus of the hippocampus in a dose-dependent manner in adult female rats. Sex differences in the effects of estradiol on hippocampal neurogenesis have been observed. Repeated estradiol exposure was found to increase cell proliferation in female rats but had no effect on male rats in one study. Both estrogen receptors, ERa and ERβ can contribute to estrogen-induced neuroprotection. In particular, the ERβ has been found to have a key role in estrogen induced neurogenesis. A number of naturally occurring ERβ selective phytoestrogens have been identified and multiple ERβ selective ligands have been synthesized. Compounds which can stimulate the estrogen receptors and consequently produce neurogenesis enhancer and/or neurotrophic effects include agonists of the estrogen receptor such as Estrone, Estriol, Estradiol, 17β-Estradiol, ICI, 182,780, Comestrol, Nonylphenol, Sah 58-035, Faslodex, and Phytoestrogens. Additionally, Estrogen agonists/antagonists may have therapeutic neurotrophic and/or neurogenesis effects such as CHF 4056, CHF 4227, and Resveratrol. Additionally selective estrogen receptor modulators may have therapeutic neurotrophic and/or neurogenesis enhancer effects such as Tamoxifen, Raloxifene, and SP500263.

Testosterone injections have been shown to result in a significant increase in neurogenesis in a dose-dependent manner in male rats. Also, one of the major metabolites of testosterone, dihydrotestosterone (DHT), resulted in a significant increase in hippocampal neurogenesis. These results have indicated that testosterone enhances hippocampal neurogenesis via increased cell survival in the dentate gyri through an androgen-dependent mechanism. Androgen receptor agonists which may enhance neurogenesis include Testosterone, Dihydrotestosterone, Methyltestosterone, and Acetothiolutamide. In addition, androgen receptor modulators may enhance neurogenesis and/or have neurotrophic effects, such as LGD-3303. Additionally selective androgen receptor modulators may exert these therapeutic effects such as the Propionamides such as C-6.

The neurogenesis enhancer according to the invention may further include secondary enhancers such as DHEA (dehydroepiandrosterone), which stimulates neurogenesis in the hippocampus of rats and promotes survival of newly formed neurons, the secondary enhancers being substances that by themselves may not improve brain function, but may have benefits for those who lack them (in the case of hormones) or may alter the balance of neurotransmitters. Enhancers which work through undiscovered mechanisms, such as Royal Jelly may also be employed under the scope of the present invention. Royal Jelly increases brain growth and diversity and has been reported to stimulate the growth of glial cells and neural stem cells in the brain. Still further, neurogenesis enhancers may include sodium ferulate and EGb 761. In yet another embodiment, direct brain stimulation may be used as a treatment according to the invention and may include electroconvulsive therapy, other brain electrical stimulation, and/or deep brain stimulation. Both electrical stimulation and deep brain stimulation have been shown to enhance neurogenesis. For example, electrical stimulation of the anterior thalamus in rats resulted in increased hippocampal neurogenesis. Also high frequency deep brain stimulation of the anterior thalamic nucleus in mice resulted in neural progenitors in the dentate gyrus, later manifested as an increased number of new neurons.

In another embodiment, the neurogenesis enhancer may include non-steroidal anti-inflammatory drugs (NSAIDs). Specifically, those skilled in the art will understand that microglia respond to pathological events such as injury or disease by becoming activated releasing pro-inflammatory cytokines. Activation of microglia in Alzheimer's disease has been shown to inhibit the brain's reparative abilities. Increased microglial activation in the dentate gyms of the hippocampus decreases the number of newly generated neurons. When systemic inflammation is inhibited by the administration of the NSAID indomethacin, there is increased hippocampal neurogenesis. Administration of indomethacin following cranial radiation decreases microglial activation and this correlates with increased neurogenesis. Exemplary NSAIDs according to the invention include Indomethacin. NSAIDs are a group of heterogeneous compounds. Originally described as COX (cyclooxygenase) inhibitors, NSAIDs might affect a multitude of signaling pathways and cellular mechanisms. These NSAID compounds impact brain inflammation through their actions on microglial cells. Also NSAIDs share the ability to inhibit the activity of the prostaglandin biosynthetic enzymes and the COX isoforms 1 and/or 2. Microglia are an important source of prostaglandins Genetic ablation or pharmacologic inhibition of COX-1 was shown to reduce inflammation and neurodegeneration in intracerebrally injected mice. COX-2 inhibitors can also mediate microglial activation and secondary cell death in at least one model. Specifically, reducing COX-2 activity can mitigate the secondary and progressive loss of dopaminergic neurons induced by MPTP (1-methyl-4-phenyl-1,2,3,6-tetrahydropyridine), possibly by suppression of microglial activation in the substantia nigra pars compacta.

Compounds that can produce full inhibition of both COX-1 and COX-2 with poor COX-2 selectivity include the following: 6MNA, Aspirin, Carprofen, Diclofenac, Fenoprofen,

Flufenarn ate, Flubiprofen, Ibuprofen, Indomethacin, Ketoprofen, Ketorolac, Meclofenamate, Mefenamic Acid, Naproxen, Niflumic Acid, Piroxicam, Sulindac Sulphide, Suprofen, Tenidap, Tolmetin, Tomoxiprol and Zomipirac. Compounds that can produce full inhibition of COX-1 and COX-2 with >5x preference towards inhibiting COX-2 include the following: Celecoxib, Etodolac, Meloxicam and Nimesulide. Compounds that appear to be only weak inhibitors of COX-1 and COX-2 include the following: Diisopropylflurophosphate, L745,337, NS398, Rofecoxib and SC58125, Other NSAID include the following: 5-Aminosalicylic acid, Ampyrone, Diflunisal, Nabumetone, Paracetamol, Resveratrol, Salicin, Salicylaldehyde, Sodium Salicylate, Sulfasalazine, Sulindac, Tamoxifen, Ticlopidine and Valeryl salicylate.

The neurogenesis enhancer may further include cannabinoids, which are known to promote both embryonic and adult hippocampus neurogenesis and to produce antidepressant-like effects. In yet another embodiment, the neurogenesis enhancer may include activation of CB1 cannabinoid receptors which increases neurogenesis and decreases anxiety-like and helpless-like behavior in two paradigms. The CB1 cannabinoid receptor activators failed to produce behavioral or neurogenic responses when radiation to the hippocampus ablated the neurogenesis response. CB1 receptor agonists (also known as central cannabinoid receptor agonists) may include HU210 (central CB1 and peripheral CB2 receptor agonist), CP 55,940 (considered a full agonist at both CB1 and CB2 receptors), WIN 55,212-2 (an agonist of CB1 and CB2 receptors, WIN 55,212-3 mesylate (a CB1 receptor partial inverse agonist and a low potency CB2 receptor silent antagonist), Δ₉-tetrahydrocannabinol (primary psychoactive component of marijuana), Δ₈-tetrahydrocannabinol (agonist of CB1 and CB2 cannabinoid receptors), ACEA (highly selective CB1 agonist), ACPA (in Tocrisolve100) (selective CB1 agonist), Arvanil (CB1 and TRPV1 agonist), (+/−)-CP 47,497 (CB1 receptor agonist), DEA (endogenous CB1 agonist), Leelamine hydrochloride (CB1 agonist), (R)-(+)-Methanandamide (selective CB1 agonist), (R)-(+)-Methanandamide (in Tocrisolve 100) (selective CB1 agonist), Noladin ether (endogenous agonist for CB1 and GPR55), Oleamide (CB1 receptor agonist), RVD-Hpa (selective CB1 agonist) and NADA (endogenous CB1 agonist). CB2 agonists, which promote neural progenitor cell proliferation and induce neural progenitor cell proliferation and neurogenesis via activation of mTORCI signaling may include HU 308 (specific agonist for CB2), GP1a (selective CB2 agonist), CB 65 (high affinity, selective CB2 agonist), GW 405833 (high affinity, CB2 receptor partial agonist), L-759,633 (high affinity, selective CB2 agonist), L-759,656 (highly selective CB2 agonist), GP 2a (selective CB2 agonist), MDA 19 (CB2 agonist), SER 601 (selective CB2 agonist) and JWH 133 (potent and selective CB2 receptor agonist).

In a further embodiment of the invention, the neurogenesis enhancer may include mood stabilizers. Mood stabilizers, like the antidepressants, produce neurotrophic effects and have been shown to enhance neurogenesis. The classical mood stabilizer, lithium, produced a significant 25% increase in BrdU-labeled cells in the dentate gyrus of the hippocampus, indicating a substantial neurogenesis effect. In addition to lithium, the other widely used mood stabilizers valproic acid and carbamazepine, have also been shown to enhance adult hippocampal neurogenesis. Another widely used mood stabilizer, lamotrigine, has been shown to up-regulate frontal and hippocampal brain derived neurotrophic factor (BDNF) expression and to restore stress induced down-regulation of BDNF expression. BDNF acts as a stimulus for neurogenesis and is more generally, a neurotrophic agent. Currently approved mood stabilizers are as follows: Valproic Acid (Depakene), Divalproex Sodium (Depakote), Lithium Carbonate (Eskalith, Lithonate), Tigabine (Gabatril), Levetiracetam (Keppra), Lamotrigine (Lamitcal), Gabapentin (Neurontin), Carbamazepine (Tegretol), Oxcarbazepine (Trileptal), Topiramate (Topamax), Zonisamide (Zonegran) and Riluzole (Rilutek).

In accordance with the exemplary method of the invention, a series of instruments were designed to better understand and measure the progression of brain aging and AD. These instruments have been helpful, worldwide, in the subsequent development of AD treatments. A pre-pilot feasibility study was conducted using two neurogenesis enhancer antidepressant agents in an effort to retard the progression of pathology and cognitive decline in the pre-MCI, SCI stage. This study was a double-blind, placebo controlled, 2 year trial using the SSRLeseitalopram (Lexapro), as well as the selective noradrenergic, and serotonegic reuptake inhibitor (SINRI), venlafaxine (Effexor) and a placebo. In this feasibility study, only the quantitatively analyzed EEG (QEEG) was employed as an imaging measure. Various QEEG measures serve as primary outcome variables. One subject completed the one year ratings in this 2 year feasibility trial.

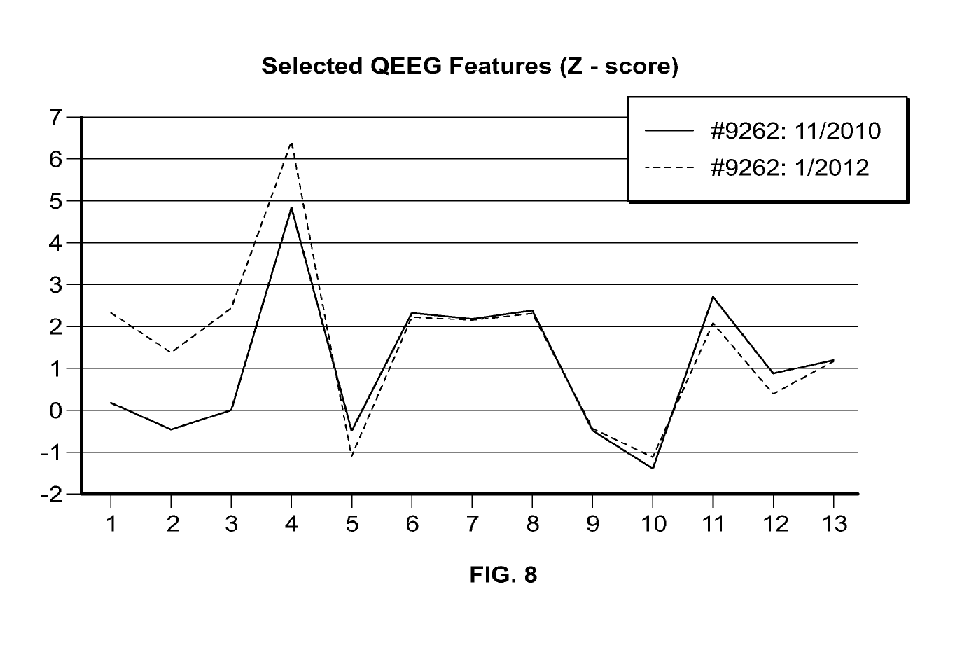

Three different EEG analytic views were obtained, as shown in FIGS. 8-11 and described in greater detail below. The first and second EEGs replicated and showed the same abnormalities. Specifically, as shown in FIG. 8 which depicts Z-scores corresponding to selected QEEG features 1-13, several of the selected features have Z-scores greater than 1.96 (p<0.05). Specifically, the selected QEEG features include Absolute Power F8 in all bands (1), Absolute Power Right Anterior Regions Theta (2), Mean Frequency P301 in Delta (3), Mean Frequency P3O1 in Theta (4), Mean Frequency C4 total spectrum (5), Relative Power (%) Anterior Regions Theta (6), Relative Power (%) Left Lateral Regions Theta (7), Relative Power (%) Right Lateral Regions Theta (8), Coherence FP1F3 Delta (9), Coherence C4P4 total spectrum (10), Anterior regions Theta (11), Posterior regions Theta (12) and Lateral regions Theta (13) wherein 1-2 are measures of absolute power, 3-5 are measures of mean frequency within the band, 6-8 are measures of relative (%) power, 9-10 are measures of coherence between regions and 11-13 are multivariate measures across regions in the theta band in Z-scores. The features having Z-scores greater than 1.96 include (1) mean frequency in left parietal occipital region in the theta band, (2) relative power abnormalities in anterior, left and right lateral regions in the theta band and (3) overall abnormalities across anterior regions, posterior regions and lateral regions in the theta band.

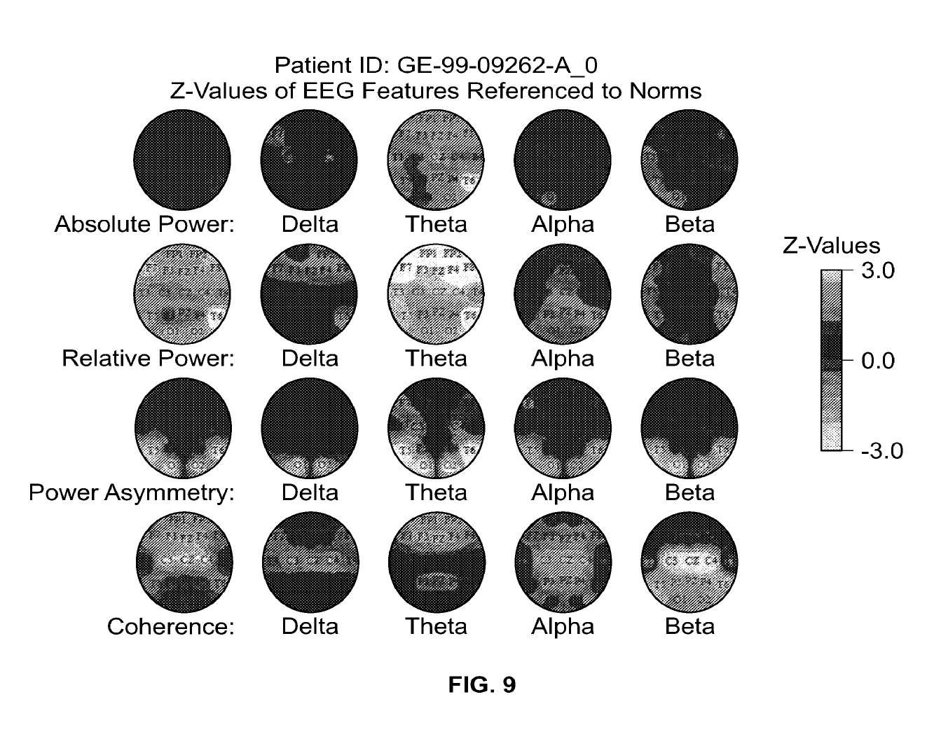

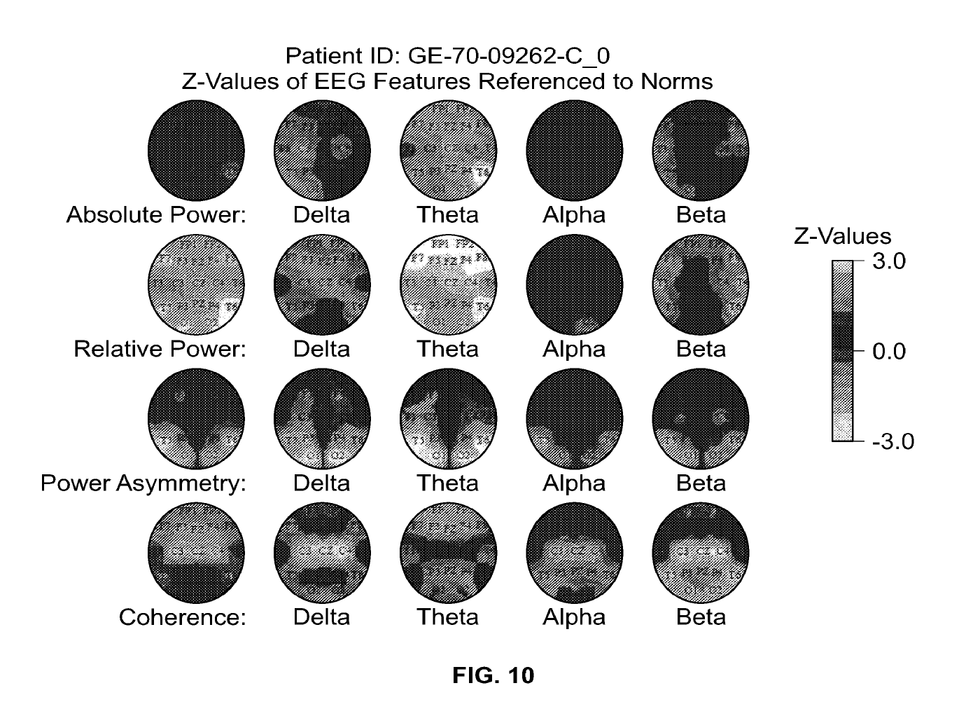

FIGS. 9-10 depict QEEG topographic images of Z-Scores representing the statistical deviation from age expected normal values for each measure set (rows) and each band (columns), wherein orientation is nose up left on left. Specifically, FIG. 9 depicts results from a baseline study while FIG. 10 depicts results from a study performed more than one year from the baseline. These topographic images indicate significant excess (p<0.01) of absolute and relative power in the theta band, with normal alpha and beta activity, significant asymmetries between occipital regions and between posterior temporal regions in all bands (R>L) and significant incoherences between central regions in beta (in both studies) and in delta (in the one year follow up study).

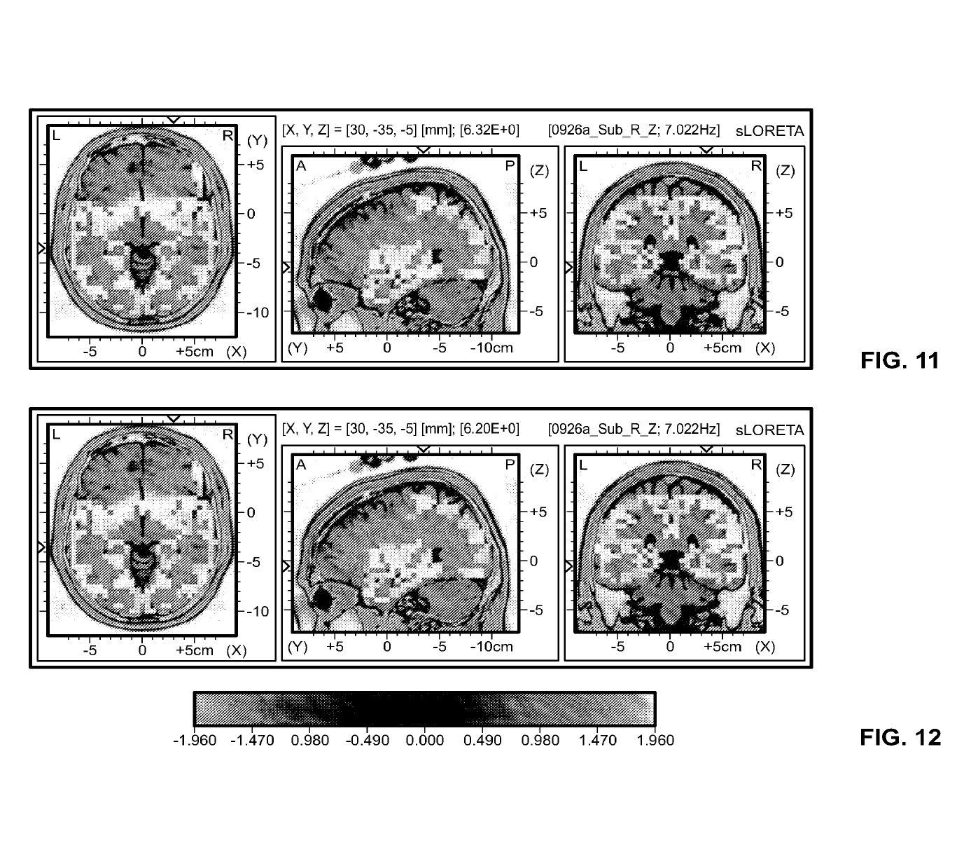

FIGS. 11-12 depict source localization (LORETA) images of the maximum abnormalities in the very narrow band frequency spectrum, color coded for significance of abnormalities (using the scale shown at the bottom of the figure, for z=±1.96 or p<0.05. The LORETA source images at maximum abnormality in the narrow band frequency spectrum, show significant abnormalities, including: significant abnormal activation in the theta band with mathematically most probable sources seen in regions including: hippocampus, parahippocampus, inferior parietal lobule, cuneus, pre-cuneus, and superior temporal gyrus. Specifically, FIG. 11 depicts a baseline LORETA image of the subject while FIG. 12 depicts the LORETA image of the subject more than one year after baseline,

The study described above demonstrates the correlative effect of pharmacological treatment (i.e., the administration of Effexor in this study) to the absence of cognitive decline of the subject over the 1 year and further evidences the present inventive concept of favorable therapeutic effects from neurogenesis enhancer antidepressant medication in SCI persons,

In accordance with an exemplary method according to the invention, a neurogenesis enhancer is administered to a subject who has been determined to be in GDS Stage 2 corresponding to SCI. As disclosed in greater detail above, the administration of this treatment slows, inhibits or reverses the subject's progression into cognitive decline and, in some cases, returns the subject to a stage of having no cognitive decline. A younger age and lower Hamilton Depression Scale score is associated with a greater likelihood of remission.

Those skilled in the art will understand that various modifications may be made to the invention without departing from the spirit or scope thereof. Thus, the present invention is intended to encompass all modifications and variations within the scope of the appended claims and their equivalents.

What is claimed is:

1. A method for preventing cognitive decline, comprising the steps of:
identifying, in an individual, a first stage of cognitive decline corresponding to Subjective Cognitive Impairment; and
administering a predetermined treatment to the individual to inhibit a progression of the individual to a second predetermined stage of cognitive decline or to inhibit progression of cognitive decline within the first stage.

2. The method of claim 1, wherein the second stage is Mild Cognitive Impairment.

3. The method of claim 1, wherein the second stage is progression into dementia.

4. The method of claim 1, wherein the second stage is Alzheimer's disease, cerebrovascular dementia, frontotemporal dementia, Lewy Body dementia, or other forms of dementia.

5. The method of claim 1, wherein the inhibition includes promoting a reversal into a stage of No Cognitive Decline.

6. The method of claim 1, wherein the treatment is a pharmacological treatment including a neurogenesis enhancer

7. The method of claim 6, wherein the treatment is one of an SSRI and a growth hormone releasing hormone.

8. The method of claim 7, wherein the SSRI is an antidepressant.

9. The method of claim 6, wherein the treatment is a combination of two or more neurogenesis enhancers.

10. The method of claim 9, wherein the treatment includes a mood stabilizer.

11. The method according to claim 9, wherein the treatment includes two or more of an antidepressant, a cholinergic, a dopaminergic, a glutamate activator, an AMPA receptor, direct hormones and cannabinoids.

12. The method of claim 1, wherein the treatment is a pharmacological treatment including at least one of an antidepressant, mood stabilizer, growth hormone releasing hormone and insulin.

13. The method of claim 1, further comprising the administration of deep brain stimulation.

14. The method of claim 1, further comprising the step of performing an analysis of a QEEG of a subject at baseline and at a predetermined period of time after treatment.

15. A method for preventing cognitive decline in a subject, comprising:
obtaining baseline subject data;
analyzing the baseline data to determined if the subject is in a first stage of cognitive decline;
administering a pharmacological treatment to the subject to inhibit cognitive decline in the subject; and
obtaining subject data at a predetermined time interval from baseline to verify that the cognitive decline has one of slowed and reversed.

16. The method of claim 15, wherein the treatment is a pharmacological treatment including a neurogenesis enhancer.

17. The method of claim 16, wherein the treatment further comprising the administration of a treatment including one of exercise and deep brain stimulation.

18. The method of claim 15, wherein the subject data includes one or more of a quantitative electroencephalogram (QEEG), low resolution electromagnetic topographic analysis (LORETA) and a Hamilton Depression Score.
